(19) United States
(12) Patent Application Publication
(10) Pub. No.: US 2026/0355183 A1
(43) Pub. Date: Nov. 12, 2026

(54) METHODS OF TREATING HEPATIC-ONLY METASTATIC UVEAL MELANOMA

(71) Applicants: IDEAYA BIOSCIENCES, INC., South San Francisco, CA (US); PFIZER INC., New York, NY (US)

(72) Inventors: MATTHEW ANTHONY MAURER, South San Francisco, CA (US); YUJIRO HATA, South San Francisco, CA (US); MICHAEL GABRIEL O'QUIGLEY, South San Francisco, CA (US)

(21) Appl. No.: 19/476,573

(22) PCT Filed: Apr. 19, 2024
(86) PCT No.: PCT/US2024/025461

§ 371 Date: Oct. 17, 2025

Related U.S. Application Data
(60) Provisional application No. 63/497,590, filed on Apr. 21, 2023.

(51) Int. Cl. A61K 31/497; A61K 31/4545; A61P 35/04

(52) U.S. Cl. CPC A61K 31/497; A61K 31/4545; A61P 35/04

(57) ABSTRACT

Provided herein is a method of treating metastatic uveal melanoma (MUM) in a patient having a hepatic tumor and an essential absence of an extra-hepatic tumor with a PKC inhibitor and a c-MET inhibitor.

### CROSS-REFERENCES TO RELATED APPLICATIONS

This application claims benefit to U.S. Provisional Application No. 63/497,590 filed Apr. 21, 2023, which is incorporated herein in its entirety and for all purposes.

### BACKGROUND

Uveal melanoma is the most common primary intraocular malignant tumor in adults. Certain protein kinase inhibitors are described in International Publication Nos. WO 02/38561 and WO 2008/106692. One protein kinase C (PKC) inhibitor, sotrastaurin, has been shown to have activity against certain PKC isototypes and has only recently been shown to selectively inhibit the growth of uveal melanoma cells harboring GNAQ mutations by targeting PKC/ERK1/2 and PKC/NF-xB pathways (see X. Wu, et al., Mol. Cancer Ther., Vol. 11, pages 1905-1914, 2012). However, there still remains an unmet need to provide next generation PKC inhibitors for treating uveal melanoma that have improved efficacy at lower dosage amounts to achieve tumor regression, improved potency, hERG activity, absorption, gastrointestinal tolerance and kinase selectivity. PCT Application No. PCT/IB2015/055951 (published as WO 2016/020864) discloses a number of potent and selective PKC inhibitors.

The c-MET receptor has been shown to be expressed in a number of human cancers. c-MET and its ligand, HGF, have also been shown to be co-expressed at elevated levels in a variety of human cancers (particularly sarcomas). However, because the receptor and ligand are usually expressed by different cell types, c-MET signaling is most commonly regulated by tumor-stroma (tumor-host) interactions. Furthermore, c-MET gene amplification, mutation, and rearrangement have been observed in a subset of human cancers. Families with germline mutations that activate c-MET kinase are prone to multiple kidney tumors as well as tumors in other tissues. Numerous studies have correlated the expression of c-MET and/or HGF/SF with the state of disease progression of different types of cancer (including lung, colon, breast, prostate, liver, pancreas, brain, kidney, ovaries, stomach, skin, and bone cancers). Furthermore, the overexpression of c-MET or HGF has been shown to correlate with poor prognosis and disease outcome in a number of major human cancers including lung, liver, gastric, and breast. c-MET has also been directly implicated in cancers without a successful treatment regimen such as pancreatic cancer, glioma, and hepatocellular carcinoma. PCT Application No. PCT/IB2005/002837 (published as WO 2006/021884) discloses a number of potent and selective c-MET inhibitors.

Uveal melanoma has an associated approximate 40% risk of metastasizing to the liver within 10 years of diagnosis of the primary tumor. Hepatic metastases, which occur in 95% of patients with metastatic uveal melanoma, result in death in almost all cases. It is therefore important to understand the pathology of metastatic uveal melanoma (MUM) to the liver in order to develop rational treatment protocols.

### SUMMARY

Provided herein is a method of treating metastatic uveal melanoma (MUM) in a patient having a hepatic tumor and an essential absence of an extra-hepatic tumor, the method comprising administering a therapeutically effective amount of a PKC inhibitor and administering a therapeutically effective amount of a cMet inhibitor to the patient.

Also provided herein is a method of treating metastatic uveal melanoma (MUM) in a patient, the method comprising:
a) selecting a patient having a hepatic tumor and an essential absence of an extra-hepatic tumor; and
b) administering a therapeutically effective amount of a PKC inhibitor and administering a therapeutically effective amount of a cMet inhibitor to the patient.

Finally, provided herein is a method of treating metastatic uveal melanoma (MUM) in a patient, the method comprising:
a) selecting a patient having a hepatic tumor and essential absence of an extra-hepatic tumor, wherein the patient selection is determined by assessing a screening test; and
b) administering a therapeutically effective amount of a PKC inhibitor and a therapeutically effective amount of a cMet inhibitor to the patient.

In some embodiments, the PKC inhibitor is Compound 1 (darovasertib). In some embodiments, the cMET inhibitor is Compound 2 (crizotinib).

### DETAILED DESCRIPTION

Provided herein is a method of treating metastatic uveal melanoma (MUM) in a patient having a hepatic tumor and an essential absence of an extra-hepatic tumor, the method comprising administering a therapeutically effective amount of a PKC inhibitor and administering a therapeutically effective amount of a cMet inhibitor to the patient.

### DEFINITIONS

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods. For example, a dose of about 300 mg may be understood to mean that the dose may vary between 270 mg and 330 mg.

As used in the specification and in the claims, the term “comprising” may include the embodiments “consisting of” and “consisting essentially of.” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated compounds, which allows the presence of only the named compounds, along with any pharmaceutically acceptable carriers, and excludes other compounds.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a dose range of “about 200 mg to about 600 mg” should be interpreted to include not only the explicitly recited concentration of about 200 mg to about 600 mg, but also include individual dosage (e.g., 250 mg, 400 mg, 550 mg) and the sub-ranges (e.g., 250 mg to 450 mg) within the indicated range. To further illustrate, a tumor size reduction of “30%-50%” should be interpreted to include not only the explicitly recited concentration of about 30% to about 50%, but also include individual percentages (e.g., 35%, 40%, 50%) and the sub-ranges (e.g., 35%-45%) within the indicated range. The term “about” can include ±1%, ±2%, ±3%, ±4%, ±5%, ±6%, ±7%, ±8%, ±9%, or ±10%, of the numerical value(s) being modified. In addition, the phrase “about ‘x’ to ‘y’” includes “about ‘x’ to about ‘y’”.

The terms “combination,” “therapeutic combination,” “pharmaceutical combination,” or “combination product” as used herein refer to either a fixed combination in one dosage unit form, or non-fixed combination in separate dosage forms, or a kit of parts for the combined administration where two or more therapeutic agents may be administered independently, at the same time or separately within time intervals.

As used herein, the term “non-fixed combination” means that the active ingredients, e.g., Compound 1 and Compound 2, are both administered to a patient as separate entities either simultaneously, concurrently or sequentially, wherein such administration provides therapeutically effective levels of the two compounds in the body of the patient.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single formulation having a fixed ratio of active ingredients or in separate formulations (i.e., in separate dosage units, for example, separate tablets, capsules and/or intravenous formulations) for each active ingredient. In addition, such administration also encompasses the use of each type of therapeutic agents in a sequential or separate manner, either at approximately the same time or at different times. Regardless of whether the active ingredients are administered as a single formulation or in separate formulations, the agents are administered to the same patient as part of the same course of therapy. The agents may be administered at the same point in time or immediately following one another. The agents may be administered at any order. The agents may be administered separately at different times during the course of therapy in such time intervals that the combination therapy is effective in treating cancer. In any case, the treatment regimen will provide beneficial effects in treating the conditions or disorders described herein.

As used herein, the term “free base equivalent” refers to the amount of the active agent (e.g., Compound 1 or Compound 2) present in the active agent or pharmaceutically acceptable salt thereof. Stated alternatively, the term “free base equivalent” means either an amount of Compound 1 or Compound 2 free base, or the equivalent amount of Compound 1 or Compound 2 free base that is provided by a salt of said compound.

As used herein, “metastasis” or “metastatic” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

As used herein, the phrase “essential absence” indicates that there was no detectable presence of a tumor as determined by common techniques in the art of cancer therapy and diagnostics.

As used herein, “Progression free survival (PFS)” for a patient is defined as the number of days from the first date of treatment to the date of the first documented disease progression (Progressive Disease (PD)) or date of death, whichever occurs first.

As used herein, “Progressive Disease (PD),” in a patient is defined as at least a 20% increase in the sum of diameters of target lesions, taking as reference the smallest sum, or nadir measurement on study. This includes the baseline sum if that is the smallest sum measured. There could also be a new lesion, or a non-target lesion that has unequivocal progression.

As used herein, “Median progression free survival (mPFS)” is defined as the length of time from start of treatment that half of the patients in a group have not progressed. (Kaplan-Meier (KM) methods are used to determine).

As used herein, “Disease control rate (DCR)” is the proportion of patients (in percentage) in the respective analysis population who have complete response (CR), partial response (PR) or stable disease (SD) lasting greater than or equal to approximately 2 months. The DCR is the sum of the CR, PR, and SD rates.

The phrase “Complete response (CR)” in a patient is defined as the disappearance of all target lesions.

The phrase “Partial response (PR)” in a patient is defined as at least a 30% decrease in the sum of diameters of target lesions, taking as reference the baseline sum diameters.

As used herein, “Confirmed Partial response (cPR)” in the clinical setting requires confirmation at least 28 days after the initial response.

As used herein, “Stable Disease (SD)” in a patient is defined as neither sufficient shrinkage to qualify for a partial response (PR) nor sufficient increase to qualify for progressive disease (PD), taking as reference the smallest sum diameters while on study.

As used herein, “Overall response rate (ORR)” is the proportion of patients in the analysis population who have complete response (CR) or partial response (PR) . . . .

As used herein, “uPR” is unconfirmed partial response.

As used herein, the term “treating” or “treatment” refers to inhibiting a disease; for example, inhibiting a disease, condition, or disorder in an individual who is experiencing or displaying the pathology or symptomology of the disease, condition, or disorder (i.e., arresting further development of the pathology and/or symptomology) or ameliorating the disease; for example, ameliorating a disease, condition, or disorder in an individual who is experiencing or displaying the pathology or symptomology of the disease, condition, or disorder (i.e., reversing the pathology and/or symptomology) such as decreasing the severity of the disease.

As used herein, the term “patient,” “individual,” or “subject” refers to a human or a non-human mammal. Non-human mammals include, for example, livestock and pets, such as ovine, bovine, porcine, canine, feline and marine mammals. Preferably, the patient, subject, or individual is human.

As used herein, the term “First-Line MUM patients” refers to patients that were not given prior systemic treatment in the metastatic setting including no prior embolization, no radiation to the metastatic sites or ablation to the liver lesions.

As used herein, the term “Any-Line MUM patients” refers to all patients that meet the patient eligibility criteria in the clinical trial as disclosed herein. Any-line MUM patients include (1) First-line MUM patients and (2) patients who had received prior therapies other than the combination (Compound 1 and Compound 2) as disclosed herein.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein a parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts described herein include the conventional non-toxic salts of the parent compound formed, for example, from non-toxic inorganic or organic acids. The pharmaceutically acceptable salts discussed herein can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are used. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound with a pharmaceutically acceptable carrier. The pharmaceutical composition facilitates administration of the composition to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound disclosed herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of a compound disclosed herein, and are physiologically acceptable to the patient. Supplementary active compounds may also be incorporated into the compositions. The “pharmaceutically acceptable carrier” may further include a pharmaceutically acceptable salt of the compound(s) disclosed herein. Other additional ingredients that may be included in the pharmaceutical compositions are known in the art and described, for example, in Remington's Pharmaceutical Sciences (Genaro, Ed., Mack Publishing Co., 1985, Easton, PA), which is incorporated herein by reference.

As used herein “RECIST 1.1” refers to Response Evaluation Criteria In Solid Tumors (RECIST) guidelines version 1.1. See. Eisenhauer et al. New response evaluation criteria in solid tumours: revised RECIST guideline (version 1.1). Eur J Cancer. 2009 January; 45(2):228-47. Reduction in tumor size of a patient is defined as tumor size reduction as measured using RECIST 1.1 criteria.

The term “single formulation” as used herein refers to a single carrier or vehicle formulated to deliver effective amounts of both therapeutic agents to a patient. The single vehicle is designed to deliver an effective amount of each of the agents, along with any pharmaceutically acceptable carriers or excipients. In some embodiments, the vehicle is a tablet, capsule, pill, or a patch. In other embodiments, the vehicle is a solution or a suspension.

The term “unit dose” is used herein to mean simultaneous administration of both agents together, in one dosage form, to the patient being treated. In some embodiments, the unit dose is a single formulation. The term “a unit dose,” as used herein can also refer to the simultaneous administration of both agents separately, in two dosage forms, to the patient being treated. In certain embodiments, the unit dose includes one or more vehicles such that each vehicle includes an effective amount of at least one of the agents along with pharmaceutically acceptable carriers and excipients. In some embodiments, the unit dose is one or more tablets, capsules, pills, or patches administered to the patient at the same time.

The dose amounts (for Compound 1 and Compound 2) are expressed as free base equivalent amounts, unless indicated otherwise.

The combination of agents described herein may display a synergistic effect. See, for example, Wagle, M-C., et al., Preclinical evaluation of PKC and MET inhibitor combination in primary and metastatic uveal melanoma, AACR Meeting 2021, the entire content of which is hereby incorporated by reference. The term “synergistic effect” as used herein, refers to action of two agents such as, for example, Compound 1 and Compound 2, producing an effect, for example, slowing the symptomatic progression of cancer or symptoms thereof, which is greater than the simple addition of the effects of each drug administered by themselves. A synergistic effect can be calculated, for example, using suitable methods such as the Sigmoid-Emax equation (Holford, N. H. G. and Scheiner, L. B., Clin. Pharmacokinet. 6:429-453 (1981)), the equation of Loewe additivity (Loewe, S. and Muischnek, H., Arch. Exp. Pathol Pharmacol. 114:313-326 (1926)) and the median-effect equation (Chou, T. C. and Talalay, P., Adv. Enzyme Regul. 22:27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

As used herein, the term “synergy” refers to the effect achieved when the active ingredients, i.e., Compound 1 and Compound 2, used together is greater than the sum of the effects that results from using the compounds alone.

In an embodiment, provided herein is a combination therapy comprising an effective amount of Compound 1 and Compound 2. An “effective amount” of a combination of agents (i.e., Compound 1 and Compound 2) is an amount sufficient to provide an observable improvement over the baseline clinically observable signs and symptoms of the disorders treated with the combination.

An “oral dose form” includes a unit dose form prescribed or intended for oral administration.

As used herein the term “normal lactate dehydrogenase (LDH) level” means a range of LDH between 40-250 Units/Liter.

### Methods of Treatment

In an aspect, provided herein is a method of treating metastatic uveal melanoma (MUM) in a patient having a hepatic tumor and an essential absence of an extra-hepatic tumor, the method comprising administering a therapeutically effective amount of a PKC inhibitor and administering a therapeutically effective amount of a cMet inhibitor to the patient.

In another aspect, provided herein is a method of treating metastatic uveal melanoma (MUM) in a patient, the method comprising:
a) selecting a patient having a hepatic tumor and an essential absence of an extra-hepatic tumor; and
b) administering a therapeutically effective amount of a PKC inhibitor and administering a therapeutically effective amount of a cMet inhibitor to the patient.

In an embodiment of the foregoing methods, the patient is selected by assessing a screening test.

In yet another aspect, provided herein is a method of treating metastatic uveal melanoma (MUM) in a patient, the method comprising:
a) selecting a patient having a hepatic tumor and an essential absence of an extra-hepatic tumor, wherein the patient selection is determined by assessing a screening test; and
b) administering a therapeutically effective amount of a PKC inhibitor and a therapeutically effective amount of a cMet inhibitor to the patient.

In another embodiment of the foregoing methods, the PKC inhibitor is Compound 1:

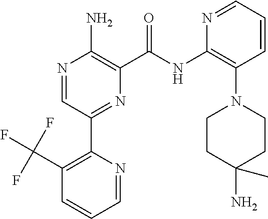 [SMILES: CC1(N)CCN(c2cccnc2NC(=O)c2nc(-c3ncccc3C(F)(F)F)cnc2N)CC1]

or a pharmaceutically acceptable salt thereof.

In yet another embodiment, the cMet inhibitor is Compound 2:

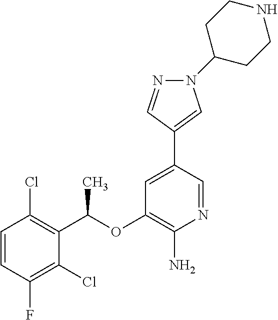 [SMILES: C[C@@H](Oc1cc(-c2cnn(C3CCNCC3)c2)cnc1N)c1c(Cl)ccc(F)c1Cl]

or a pharmaceutically acceptable salt thereof.

In any one of embodiments of the foregoing methods, the patient has a hepatic tumor but does not have an extra-hepatic tumor.

In any one of embodiments of the foregoing methods, the patient is human. In some embodiments, the patient is an adult of at least 18 years of age.

In some embodiments, the treatment reduces the size of the hepatic tumor by at least about 10%. In some embodiments, the treatment reduces the size of the hepatic tumor by at least about 20%. In some embodiments, the treatment reduces the size of the hepatic tumor by at least about 30%. In some embodiments, the treatment reduces the size of the hepatic tumor by at least about 40%. In some embodiments, the treatment reduces the size of the hepatic tumor by at least about 50%.

In some embodiments, the treatment reduces the size of the hepatic tumor by at least 10%. In some embodiments, the treatment reduces the size of the hepatic tumor by at least 20%. In some embodiments, the treatment reduces the size of the hepatic tumor by at least 30%. In some embodiments, the treatment reduces the size of the hepatic tumor by at least 40%. In some embodiments, the treatment reduces the size of the hepatic tumor by at least 50%.

In some embodiments, the treatment reduces the hepatic tumor in a size of from about 10% to about 70%. In some embodiments, the treatment reduces the hepatic tumor in a size of from about 10% to about 60%. In some embodiments, the treatment reduces the hepatic tumor in a size of from about 10% to about 50%. In some embodiments, the treatment reduces the size of hepatic tumor in an average of at least about 30%.

In some embodiments, the treatment reduces the hepatic tumor in a size of from 10% to 70%. In some embodiments, the treatment reduces the hepatic tumor in a size of from 10% to 60%. In some embodiments, the treatment reduces the hepatic tumor in a size of from 10% to 50%. In some embodiments, the treatment reduces the size of hepatic tumor in an average of at least 30%.

In an embodiment, the patient has a progression free survival of greater than or equal to 3 months. In another embodiment, the patient has a progression free survival of greater than or equal to 4, 5, 6, 7, or 8 months. In yet another embodiment, the patient has a progression free survival of greater than or equal to 5 months. In yet another embodiment, the patient has a progression free survival of greater than or equal to 6 months. In still another embodiment, the patient has a progression free survival of greater than or equal to 7 months. In yet another embodiment, the patient has a progression free survival of greater than or equal to 8 months. In an embodiment, the patient has a progression free survival of greater than or equal to 9 months. In another embodiment, the patient has a progression free survival of greater than or equal to 10 months. In yet another embodiment, the patient has a progression free survival of greater than or equal to 11 months. In still another embodiment, the patient has a progression free survival of greater than or equal to 3, 5, 7, 9, 10, or 11 months.

In some embodiments, the treatment has a disease control rate of at least about 60%, about 70%, or about 80%. In some embodiments, the treatment has a disease control rate of at least about 85%. In some embodiments, the treatment has a disease control rate of at least about 90%. In some embodiments, the treatment has a disease control rate of at least about 92%. In some embodiments, the treatment has a disease control rate of at least about 95%. In some embodiments, the treatment has a disease control rate of at least about 98%. In some embodiments, the treatment has a disease control rate of at least about 99%. In some embodiments, the treatment has a disease control rate of about 100%.

In another embodiment, the treatment has a disease control rate of at least 60%, 70%, or 80%. In yet another embodiment, the treatment has a disease control rate of at least 85%. In still another embodiment, the treatment has a disease control rate of at least 90%. In an embodiment, the treatment has a disease control rate of at least 92%. In another embodiment, the treatment has a disease control rate of at least 95%. In yet another embodiment, the treatment has a disease control rate of at least 98%. In still another embodiment, the treatment has a disease control rate of at least 99%. In some embodiments, the treatment has a disease control rate of 100%.

In an embodiment, Compound 1, or an equivalent dose of a pharmaceutically acceptable salt thereof, is administered at a dosage of about 400 mg to about 600 mg daily.

In some embodiments, Compound 1, or an equivalent dose of a pharmaceutically acceptable salt thereof, is administered in a total daily dosage of about 400 mg to about 600 mg. In some embodiments, Compound 1, or an equivalent dose of a pharmaceutically acceptable salt thereof, is administered in a total daily dosage of 400 mg to 600 mg.

In some embodiments, Compound 1, or an equivalent dose of a pharmaceutically acceptable salt thereof, is administered in a total daily dosage of about 200 mg to about 600 mg. In some embodiments, Compound 1, or an equivalent dose of a pharmaceutically acceptable salt thereof, is administered in a total daily dosage of 200 mg to 600 mg.

In another embodiment, Compound 1, or an equivalent dose of a pharmaceutically acceptable salt thereof, is administered at a dose of about 300 mg two times per day (BID). In some embodiments, Compound 1, or an equivalent dose of a pharmaceutically acceptable salt thereof, is administered at a dose of 300 mg two times per day (BID).

In yet another embodiment, Compound 1, or an equivalent dose of a pharmaceutically acceptable salt thereof, is administered at a dose of about 200 mg two times per day (BID). In some embodiments, Compound 1, or an equivalent dose of a pharmaceutically acceptable salt thereof, is administered at a dose of 200 mg two times per day (BID). In some embodiments, Compound 1, or an equivalent dose of a pharmaceutically acceptable salt thereof, is administered at a dose of 150 mg two times per day (BID). In some embodiments, Compound 1, or an equivalent dose of a pharmaceutically acceptable salt thereof, is administered at a dose of 100 mg two times per day (BID).

In still another embodiment, Compound 2, or an equivalent dose of a pharmaceutically acceptable salt thereof, is administered at a dose of about 400 mg to about 500 mg daily.

In some embodiments, Compound 2, or an equivalent dose of a pharmaceutically acceptable salt thereof, is administered in a total daily dosage of about 400 mg to about 500 mg. In some embodiments, Compound 2, or an equivalent dose of a pharmaceutically acceptable salt thereof, is administered in a total daily dosage of 400 mg to 500 mg.

In an embodiment, Compound 2, or an equivalent dose of a pharmaceutically acceptable salt thereof, is administered at a dose of about 200 mg two times per day (BID). In some embodiments, Compound 2, or an equivalent dose of a pharmaceutically acceptable salt thereof, is administered at a dose of 200 mg two times per day (BID).

In some embodiments, Compound 2, or an equivalent dose of pharmaceutically acceptable salt thereof, is administered daily. In some embodiments, Compound 2, or an equivalent dose of pharmaceutically acceptable salt thereof, is administered every other day (e.g., administered on day 1 and not administered on day 2). In some embodiments, Compound 2, or an equivalent dose of pharmaceutically acceptable salt thereof, is administered every 3 days (e.g., administered on day 1 and not administered on days 2-3).

In some embodiments, Compound 1, or an equivalent dose of a pharmaceutically acceptable salt thereof, and Compound 2, or an equivalent dose of a pharmaceutically acceptable salt thereof, are each administered daily. In some embodiments, Compound 1, or an equivalent dose of a pharmaceutically acceptable salt thereof, and Compound 2, or an equivalent dose of a pharmaceutically acceptable salt thereof, are independently administered every other day (e.g., on day 1, Compounds 1 and 2 are both administered, and on day 2, Compounds 1 and 2 are not administered; or on day 1, Compound 1 is administered and Compound 2 is not administered, and on day 2, Compound 1 is not administered and Compound 2 is administered). In some embodiments, Compound 1, or an equivalent dose of a pharmaceutically acceptable salt thereof, and Compound 2, or an equivalent dose of a pharmaceutically acceptable salt thereof, are independently administered every 3 days, wherein Compound 1 and Compound 2 are administered on the same day or on a different day.

In some embodiments, the treatment is according to a dosing schedule comprising
(i) a first treatment cycle of at least one 7-day dosing cycle, wherein Compound 1, or an equivalent dose of a pharmaceutically acceptable salt thereof, is administered alone at a dose of about 300 mg BID every day of the first treatment cycle, followed by
(ii) a second treatment cycle of at least one 7-day dosing cycle, wherein Compound 1, or an equivalent dose of a pharmaceutically acceptable salt thereof, is administered at a dose of about 300 mg BID, and Compound 2, or an equivalent dose of a pharmaceutically acceptable salt thereof, is administered at a dose of about 200 mg BID every day of the second treatment cycle.

In some embodiments, the treatment is according to a dosing schedule comprising
(i) a first treatment cycle of one 7-day dosing cycle, wherein Compound 1, or an equivalent dose of a pharmaceutically acceptable salt thereof, is administered alone at a dose of about 300 mg BID every day of the first treatment cycle, followed by
(ii) a second treatment cycle of at least three 7-day dosing cycles, wherein Compound 1, or an equivalent dose of a pharmaceutically acceptable salt thereof, is administered at a dose of about 300 mg BID, and Compound 2, or an equivalent dose of a pharmaceutically acceptable salt thereof, is administered at a dose of about 200 mg BID every day of the second treatment cycle.

In some embodiments, the treatment is according to a dosing schedule comprising
(i) a first treatment cycle of at least one 7-day dosing cycle, wherein Compound 1, or an equivalent dose of a pharmaceutically acceptable salt thereof, is administered alone at a dose of 300 mg BID every day of the first treatment cycle, followed by
(ii) a second treatment cycle of at least one 7-day dosing cycle, wherein Compound 1, or an equivalent dose of a pharmaceutically acceptable salt thereof, is administered at a dose of 300 mg BID, and Compound 2, or an equivalent dose of a pharmaceutically acceptable salt thereof, is administered at a dose of 200 mg BID every day of the second treatment cycle.

In some embodiments, the treatment is according to a dosing schedule comprising
(i) a first treatment cycle of one 7-day dosing cycle, wherein Compound 1, or an equivalent dose of a pharmaceutically acceptable salt thereof, is administered alone at a dose of 300 mg BID every day of the first treatment cycle, followed by
(ii) a second treatment cycle of at least three 7-day dosing cycles, wherein Compound 1, or an equivalent dose of a pharmaceutically acceptable salt thereof, is administered at a dose of 300 mg BID, and Compound 2, or an equivalent dose of a pharmaceutically acceptable salt thereof, is administered at a dose of 200 mg BID every day of the second treatment cycle.

In some embodiments, the second treatment cycle comprises at least seven (7) 7-day dosing cycles. In some embodiments, the second treatment cycle comprises at least eleven (11) 7-day dosing cycles. In some embodiments, the second treatment cycle comprises at least 15, 19, 23, 27, 31, 35, 39, 43, 47, 51, 55, 59, 63, 67, 71, 75, 79, 83, 87, 91, 95, 99, or 103 7-day dosing cycles.

In another embodiment,
Compound 1, or a pharmaceutically acceptable salt thereof, is administered at a dose of about 600 mg daily based on a free base equivalent of Compound 1; and
Compound 2, or a pharmaceutically acceptable salt thereof, is administered at a dose of about 400 mg daily based on a free base equivalent of Compound 2.

In some embodiments, Compound 1, or a pharmaceutically acceptable salt thereof, is administered at a total daily dosage of about 600 mg based on a free base equivalent of Compound 1; and Compound 2, or a pharmaceutically acceptable salt thereof, is administered at a total daily dosage of about 400 mg based on a free base equivalent of Compound 2.

In some embodiments, Compound 1, or a pharmaceutically acceptable salt thereof, is administered at a total daily dosage of 600 mg based on a free base equivalent of Compound 1; and Compound 2, or a pharmaceutically acceptable salt thereof, is administered at a total daily dosage of 400 mg based on a free base equivalent of Compound 2.

In some embodiments, the second treatment cycle comprises at least one 7-day dosing cycle. In some embodiments of the second treatment cycle, the 7-day dosing cycle comprises administering Compound 1, or an equivalent dose of a pharmaceutically acceptable salt thereof, and Compound 2, or an equivalent dose of a pharmaceutically acceptable salt thereof, daily. In some embodiments of the second treatment cycle, during the 7-day dosing cycle, Compound 1, or an equivalent dose of a pharmaceutically acceptable salt thereof, and Compound 2, or an equivalent dose of a pharmaceutically acceptable salt thereof, are each administered daily at each of the dosages as described in any one of forgoing embodiments.

In another embodiment,
Compound 1, or an equivalent dose of a pharmaceutically acceptable salt thereof, is administered at a dose of about 300 mg BID every day of at least one 7-day dosing cycle; and
Compound 2, or an equivalent dose of a pharmaceutically acceptable salt thereof, is administered at a dose of about 200 mg BID every day of at least one 7-day dosing cycle.

In some embodiments, Compound 1, or an equivalent dose of a pharmaceutically acceptable salt thereof, is administered daily at a dose of 300 mg BID for at least one 7-day dosing cycle; and Compound 2, or an equivalent dose of a pharmaceutically acceptable salt thereof, is administered daily at a dose of 200 mg BID for at least one 7-day dosing cycle.

In some embodiments, the treatment comprises a first treatment cycle and a second treatment cycle, each of which is described above. In some embodiments, the first treatment cycle comprises at least one 7-day dosing cycle, wherein Compound 1 or an equivalent dose of a pharmaceutically acceptable salt thereof, is administered alone. In some embodiments, the first treatment cycle comprises at least one 7-day dosing cycle, wherein Compound 1 or an equivalent dose of a pharmaceutically acceptable salt thereof, is administered alone at each of the dosages as described in any one of forgoing embodiments. In some embodiments, the second treatment cycle comprises at least seven (7) 7-day dosing cycles, wherein Compound 1, or an equivalent dose of a pharmaceutically acceptable salt thereof, and Compound 2, or an equivalent dose of a pharmaceutically acceptable salt thereof, are each administered. In some embodiments, the second treatment cycle comprises at least seven (7) 7-day dosing cycles, wherein Compound 1, or an equivalent dose of a pharmaceutically acceptable salt thereof, and Compound 2, or an equivalent dose of pharmaceutically acceptable salt thereof, are each administered at each of the dosages as described in any one of forgoing embodiments. In some embodiments, Compound 1, or an equivalent dose of a pharmaceutically acceptable salt thereof, and Compound 2, or an equivalent dose of a pharmaceutically acceptable salt thereof, are each administered for at least seven (7) 7-day dosing cycles (as described above).

In yet another embodiment, the metastatic uveal melanoma is a solid tumor. In still another embodiment, the solid tumor harbors a GNAQ or GNA11 mutation.

In an embodiment, Compound 1, or an equivalent dose of a pharmaceutically acceptable salt thereof, and Compound 2, or an equivalent dose of a pharmaceutically acceptable salt thereof, are administered in a single formulation. In another embodiment, Compound 1, or an equivalent dose of a pharmaceutically acceptable salt thereof, and Compound 2, or an equivalent dose of a pharmaceutically acceptable salt thereof, are administered in a single formulation further comprising one or more pharmaceutically acceptable carriers. In yet another embodiment, Compound 1, or an equivalent dose of a pharmaceutically acceptable salt thereof, and Compound 2, or an equivalent dose of a pharmaceutically acceptable salt thereof, are administered in a single formulation further comprising one or more pharmaceutically acceptable carriers.

In still another embodiment, Compound 1, or an equivalent dose of a pharmaceutically acceptable salt thereof, and Compound 2, or an equivalent dose of a pharmaceutically acceptable salt thereof, are administered separately.

In an embodiment, the screening test is selected from CT scan, MRI, PET scan, ultrasound, and X-ray, or a combination thereof. In an embodiment, the screening test is CT scan (also called CAT scan or computed tomography scan). In an embodiment, the screening test is MRI (Magnetic Resonance Imaging). In an embodiment, the screening test is PET scan (Positron Emission Tomography). In an embodiment, the screening test is ultrasound. In an embodiment, the screening test is X-ray.

In yet another embodiment, the method of treating MUM can further comprise: continuing to administer Compound 1, or an equivalent dose of a pharmaceutically acceptable salt thereof, and Compound 2, or an equivalent dose of a pharmaceutically acceptable salt thereof, uninterrupted during a dosing schedule comprising at least two or more consecutive 7-day dosing cycles, or preferably at least four or more consecutive 7-day dosing cycles. In some embodiments, the treatment is interrupted followed by restarting the treatment according to dosing schedules disclosed herein.

In yet another embodiment, the treatment is according to a dosing schedule comprising
(i) a first treatment cycle of one 7-day dosing cycle, wherein Compound 1, or an equivalent dose of a pharmaceutically acceptable salt thereof, is administered alone at a dose of about 300 mg BID every day of the first treatment cycle, followed by
(ii) a second treatment cycle of at least three 7-day dosing cycles, wherein Compound 1, or an equivalent dose of a pharmaceutically acceptable salt thereof, is administered at a dose of about 300 mg BID, and Compound 2, or an equivalent dose of a pharmaceutically acceptable salt thereof, is administered at a dose of about 200 mg BID every day of the second treatment cycle, wherein the treatment is uninterrupted during the second treatment cycle of at least three 7-day dosing cycles or at least seven (7) 7-day dosing cycles.

In yet another embodiment, the treatment is according to a dosing schedule comprising
(i) a first treatment cycle of one 7-day dosing cycle, wherein Compound 1, or an equivalent dose of a pharmaceutically acceptable salt thereof, is administered alone at a dose of 300 mg BID every day of the first treatment cycle, followed by
(ii) a second treatment cycle of at least three 7-day dosing cycles, wherein Compound 1, or an equivalent dose of a pharmaceutically acceptable salt thereof, is administered at a dose of 300 mg BID, and Compound 2, or an equivalent dose of a pharmaceutically acceptable salt thereof, is administered at a dose of 200 mg BID every day of the second treatment cycle,
   wherein the treatment is uninterrupted during the second treatment cycle of at least three 7-day dosing cycles or at least seven (7) 7-day dosing cycles.

In particular embodiments, Compound 1, or an equivalent dose of a pharmaceutically acceptable salt thereof, and Compound 2, or an equivalent dose of a pharmaceutically acceptable salt thereof, are continued to be co-administered uninterrupted during a dosing schedule comprising at least eight or more, twelve or more, twenty-four or more, forty-eight or more or ninety-six or more consecutive 7-day dosing cycles. In an embodiment, Compound 1, or an equivalent dose of a pharmaceutically acceptable salt thereof, is continued to be co-administered uninterrupted during a dosing schedule comprising at least eight or more, twelve or more, twenty-four or more, forty-eight or more or ninety-six or more consecutive 7-day dosing cycles. In another embodiment, Compound 2, or an equivalent dose of a pharmaceutically acceptable salt thereof, is continued to be co-administered uninterrupted during a dosing schedule comprising at least eight or more, twelve or more, twenty-four or more, forty-eight or more or ninety-six or more consecutive 7-day dosing cycles.

Exemplary lengths of time associated with the course of the treatment methods is about five years, about, 4 years, about 3 years, about 2 years, about 1 year, about 11 months, about 10 months, about 9 months, about 8 months, about 7 months, about 6 months, about 5 months, about 4 months, about 3 months, about 2 months, or about 1 month.

Exemplary lengths of time associated with the course of the treatment methods is about five years and so on; or any days, weeks, months, or years in between; for example a treatment cycle can include 5 months and additional weeks and/or days, or one year and additional months, weeks, and/or days, and the like.

In some embodiments, Compound 1, or an equivalent dose of a pharmaceutically acceptable salt thereof, and Compound 2, or an equivalent dose of a pharmaceutically acceptable salt thereof, are administered continuously (i.e., a continuous treatment until termination).

The GNAQ or GNA11 tumor to be treated can include one or more mutations, including a substitution mutation, an insertion mutation, and/or a deletion in GNAQ or GNA11 mutation. In some embodiments, the GNAQ or GNA11 mutation is a gain of function mutation. In some embodiments, the GNAQ or GNA11 mutation activates the PKC signaling pathway. In various embodiments, the GNAQ or GNA11 mutation can be the substitution of glutamine in codon 209 (Q209) and/or a substitution of arginine in codon 183 (R183). The GNAQ or GNA11 mutation can be a substitution other than glutamine in codon 209 (Q209), other than a substitution of arginine in codon 183 (R183), or other than both. In some embodiments, the GNAQ mutation is one of Q209P, Q209L, Q209H, Q209K, or Q209Y, or the GNA11 mutation is one of Q209P, Q209L, Q209K or Q209H. In further embodiments, the GNAQ mutation can be R183Q, or the GNA11 mutation can be R183C or R183H. In yet further examples, the GNAQ or GNA11 mutation is at one or more of R256, L279, R166, A168, R210, R213, R166, A231, A342, D333, G171, R147, R73, T47, E191, E221, R149, T175, T379, T85, A86, E163, D195, E319, E191, E280, E49, P293, R300, R338, R60, D155, D205, D321, 1226, R37, or V240. In further examples, the GNAQ or GNA11 tumor can comprise one or more of a Q209P, Q209L, Q209H, Q209K, Q209Y, or R183Q mutation in GNAQ, or the GNAQ or GNA11 tumor can comprise one or more of a Q209P, Q209L, Q209H, or Q209K mutation in GNA11. Additional examples of mutations in GNAQ or GNA11 are described in WO 2020/146355, which is incorporated by reference herewith in its entirety.

Exemplary lengths of time associated with the course of the treatment methods disclosed herein include: about one week; about two weeks; about three weeks; about four weeks; about five weeks; about six weeks; about seven weeks; about eight weeks; about nine weeks; about ten weeks; about eleven weeks; about twelve weeks; about thirteen weeks; about fourteen weeks; about fifteen weeks; about sixteen weeks; about seventeen weeks; about eighteen weeks; about nineteen weeks; about twenty weeks; about twenty-one weeks; about twenty-two weeks; about twenty-three weeks; about twenty four weeks; about 4 months; about seven months; about eight months; about nine months; about ten months; about eleven months; about twelve months; about thirteen months; about fourteen months; about fifteen months; about sixteen months; about seventeen months; about eighteen months; about nineteen months; about twenty months; about twenty one months; about twenty-two months; about twenty-three months; about twenty-four months; about thirty months; about three years; about four years and about five years and so on; or any days, weeks, months, or years in between; for example a treatment cycle can include 5 months and additional weeks and/or days, or one year and additional months, weeks, and/or days, and the like.

In an embodiment, both Compound 1 and Compound 2 are administered orally.

In an embodiment of the methods, the method involves the administration of a therapeutically effective amount of a combination or composition comprising compounds provided herein, or pharmaceutically acceptable salts thereof, to a subject (including, but not limited to a human or animal) in need of treatment (including a subject identified as in need).

In another embodiment of the methods, the treatment includes co-administering the amount of Compound 1, or an equivalent dose of a pharmaceutically acceptable salt thereof, and the amount of Compound 2, or an equivalent dose of a pharmaceutically acceptable salt thereof. In an embodiment, the amount of Compound 1, or an equivalent dose of a pharmaceutically acceptable salt thereof, and the amount of Compound 2, or an equivalent dose of a pharmaceutically acceptable salt thereof, are in a single formulation or unit dose form. In still other embodiments, the amount of Compound 1, or an equivalent dose of a pharmaceutically acceptable salt thereof, and the amount of Compound 2, or an equivalent dose of a pharmaceutically acceptable salt thereof, are in separate formulations or unit dose forms.

In the foregoing methods, the treatment can include administering the amount of Compound 1, or an equivalent dose of a pharmaceutically acceptable salt thereof, and the amount of Compound 2, or an equivalent dose of a pharmaceutically acceptable salt thereof, at substantially the same time or administering the amount of Compound 1, or an equivalent dose of a pharmaceutically acceptable salt thereof, and the amount of Compound 2, or an equivalent dose of a pharmaceutically acceptable salt thereof, at different times. In some embodiments of the foregoing methods, the amount of Compound 1, or an equivalent dose of a pharmaceutically acceptable salt thereof, and/or the amount of Compound 2, or an equivalent dose of a pharmaceutically acceptable salt thereof, is administered at dosages that would not be effective when one or both of Compound 1, or an equivalent dose of a pharmaceutically acceptable salt thereof, and Compound 2, or an equivalent dose of a pharmaceutically acceptable salt thereof, is administered alone, but which amounts are effective in combination.

In one embodiment of the foregoing methods, the subject is a First-Line subject. In another embodiment, the subject is a First-Line MUM subject.

The “extra-hepatic tumor” as used herein refers to any tumor outside of liver.

### Administration/Dosage/Formulations

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could begin administration of the pharmaceutical composition to dose the disclosed compound at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of the disclosed compound calculated to produce the desired therapeutic effect in association with the required pharmaceutical vehicle. The dosage unit forms are dictated by and directly dependent on (a) the unique characteristics of the disclosed compound and the particular therapeutic effect to be achieved, and (b) the limitations inherent in the art of compounding/formulating such a disclosed compound for the treatment of pain, a depressive disorder, or drug addiction in a patient.

In one embodiment, the compounds provided herein are formulated using one or more pharmaceutically acceptable excipients or carriers. In one embodiment, the pharmaceutical compositions provided herein comprise a therapeutically effective amount of a disclosed compound and a pharmaceutically acceptable carrier.

The optimum ratios, individual and combined dosages, and concentrations of the drug compounds that yield efficacy without toxicity are based on the kinetics of the active ingredients' availability to target sites, and are determined using methods known to those of skill in the art.

Routes of administration of any of the compositions discussed herein include oral, nasal, rectal, intravaginal, parenteral, buccal, sublingual or topical. The compounds may be formulated for administration by any suitable route, such as for oral or parenteral, for example, transdermal, transmucosal (e.g., sublingual, lingual, (trans) buccal, (trans) urethral, vaginal (e.g., trans- and perivaginally), (intra) nasal and (trans) rectal), intravesical, intrapulmonary, intraduodenal, intragastrical, intrathecal, subcutaneous, intramuscular, intradermal, intra-arterial, intravenous, intrabronchial, inhalation, and topical administration. In one embodiment, the preferred route of administration is oral.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions are not limited to the particular formulations and compositions that are described herein.

For oral application, particularly suitable are tablets, dragees, liquids, drops, suppositories, or capsules, caplets and gel caps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For parenteral administration, the disclosed compounds may be formulated for injection or infusion, for example, intravenous, intramuscular or subcutaneous injection or infusion, or for administration in a bolus dose or continuous infusion. Suspensions, solutions or emulsions in an oily or aqueous vehicle, optionally containing other formulatory agents such as suspending, stabilizing or dispersing agents may be used.

### Kits

In an aspect, the present disclosure provides a kit for treating metastatic uveal melanoma (MUM) in a patient having a hepatic tumor and an essential absence of an extra-hepatic tumor, comprising Compound 1, or an equivalent dose of a pharmaceutically acceptable salt thereof, in a dosage of about 400 mg to about 600 mg, and Compound 2, or an equivalent dose of a pharmaceutically acceptable salt thereof, in a dosage of about 400 mg to about 500 mg. In another embodiment, the present disclosure provides a kit for treating metastatic uveal melanoma (MUM) in a patient having a hepatic tumor and an essential absence of an extra-hepatic tumor, comprising Compound 1, or an equivalent dose of a pharmaceutically acceptable salt thereof, in an amount of about 300 mg, and Compound 2, or an equivalent dose of a pharmaceutically acceptable salt thereof, in an amount of about 200 mg. In another embodiment, the present disclosure provides a kit for treating metastatic uveal melanoma (MUM) in a patient having a hepatic tumor and an essential absence of an extra-hepatic tumor, comprising Compound 1, or an equivalent dose of a pharmaceutically acceptable salt thereof, in an amount from about 400 mg per day to about 600 mg per day, and Compound 2, or an equivalent dose of a pharmaceutically acceptable salt thereof, in an amount from about 400 mg per day to about 500 mg per day. In some embodiments, the kit further comprises packaging and instructions.

In additional embodiments, pharmaceutical kits are provided. The kit includes a sealed container approved for the storage of pharmaceutical compositions, the container containing one of the above-described pharmaceutical compositions. In some embodiments, the sealed container minimizes the contact of air with the ingredients, e.g., an airless bottle. In other embodiments, the sealed container is a sealed tube. An instruction for the use of the composition and the information about the composition are to be included in the kit.

In a particular embodiment, the compounds of the combination can be dosed on the same schedule, whether by administering a single formulation or unit dose form containing all of the compounds of the combination, or by administering separate formulations or unit dose forms of the compounds of the combination. However, some of the compounds used in the combination may be administered more frequently than once per day, or with different frequencies that other compounds in the combination. Therefore, in one embodiment, the kit contains a formulation or unit dose form containing all of the compounds in the combination of compounds, and an additional formulation or unit dose form that includes one of the compounds in the combination of agents, with no additional active compound, in a container, with instructions for administering the dose forms on a fixed schedule.

The kits provided herein comprise prescribing information, for example, to a patient or health care provider, or as a label in a packaged pharmaceutical formulation. Prescribing information may include for example efficacy, dosage and administration, contraindication and adverse reaction information pertaining to the pharmaceutical formulation.

In all of the foregoing the combination of compounds of the invention can be administered alone, as mixtures, or with additional active agents.

A kit provided herein can be designed for conditions necessary to properly maintain the components housed therein (e.g., refrigeration or freezing). A kit can contain a label or packaging insert including identifying information for the components therein and instructions for their use (e.g., dosing parameters, clinical pharmacology of the active ingredient(s), including mechanism(s) of action, pharmacokinetics and pharmacodynamics, adverse effects, contraindications, etc.).

Each component of the kit can be enclosed within an individual container, and all of the various containers can be within a single package. Labels or inserts can include manufacturer information such as lot numbers and expiration dates. The label or packaging insert can be, e.g., integrated into the physical structure housing the components, contained separately within the physical structure, or affixed to a component of the kit (e.g., an ampule, syringe or vial).

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings of the present disclosure as set forth.

### Non-Limiting Exemplary Embodiments

Embodiment 1. A method of treating metastatic uveal melanoma (MUM) in a patient having a hepatic tumor and an essential absence of an extra-hepatic tumor, the method comprising administering a therapeutically effective amount of a PKC inhibitor and administering a therapeutically effective amount of a cMet inhibitor to the patient.

Embodiment 2. A method of treating metastatic uveal melanoma (MUM) in a patient, the method comprising:
a) selecting a patient having a hepatic tumor and an essential absence of an extra-hepatic tumor; and
b) administering a therapeutically effective amount of a PKC inhibitor and administering a therapeutically effective amount of a cMet inhibitor to the patient.

Embodiment 3. The method of embodiment 1 or 2, wherein the patient is selected by assessing a screening test.

Embodiment 4. A method of treating metastatic uveal melanoma (MUM) in a patient, the method comprising:
a) selecting a patient having a hepatic tumor and essential absence of an extra-hepatic tumor, wherein the patient selection is determined by assessing a screening test; and
b) administering a therapeutically effective amount of a PKC inhibitor and a therapeutically effective amount of a cMet inhibitor to the patient.

Embodiment 5. The method of any one of embodiments 1 to 4, wherein the PKC inhibitor is Compound 1:

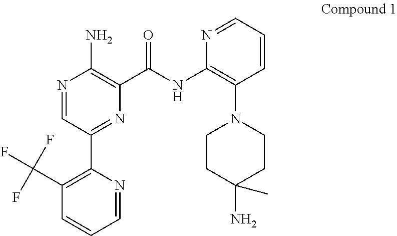 [SMILES: CC1(N)CCN(c2cccnc2NC(=O)c2nc(-c3ncccc3C(F)(F)F)cnc2N)CC1]

or a pharmaceutically acceptable salt thereof.

Embodiment 6. The method of any one of embodiments 1 to 5, wherein the cMet inhibitor is Compound 2:

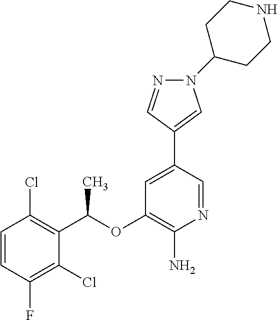 [SMILES: C[C@@H](Oc1cc(-c2cnn(C3CCNCC3)c2)cnc1N)c1c(Cl)ccc(F)c1Cl]

or a pharmaceutically acceptable salt thereof.

Embodiment 7. The method of any one of embodiments 1 to 6, wherein the patient does not have an extra-hepatic tumor.

Embodiment 8. The method of any one of embodiments 1 to 7, wherein the patient is an adult of at least 18 years of age.

Embodiment 9. The method of any one of embodiments 1 to 8, wherein the treatment reduces a size of the hepatic tumor by at least 10%.

Embodiment 10. The method of any one of embodiments 1 to 8, wherein the treatment reduces a size of the hepatic tumor by at least about 10%.

Embodiment 11. The method of any one of embodiments 1 to 10, wherein the patient has a progression free survival of greater than or equal to 3 months.

Embodiment 12. The method of any one of embodiments 1 to 11, wherein the patient has a progression free survival of greater than or equal to 4, 5, 6, 7, or 8 months.

Embodiment 13. The method of any one of embodiments 1 to 12, wherein the patient has a progression free survival of greater than or equal to 9 months.

Embodiment 14. The method of any one of embodiments 1 to 13, wherein the patient has a progression free survival of greater than or equal to 10 months.

Embodiment 15. The method of any one of embodiments 1 to 14, wherein the patient has a progression free survival of greater than or equal to 11 months.

Embodiment 16. The method of any one of embodiments 1 to 15, wherein the treatment has a disease control rate of at least 60%, 70%, or 80%.

Embodiment 17. The method of any one of embodiments 1 to 16, wherein the treatment has a disease control rate of at least 85%.

Embodiment 18. The method of any one of embodiments 1 to 17, wherein the treatment has a disease control rate of at least 90%.

Embodiment 19. The method of any one of embodiments 1 to 18, wherein the treatment has a disease control rate of at least 92%.

Embodiment 20. The method of any one of embodiments 1 to 19, wherein the treatment has a disease control rate of at least 95%.

Embodiment 21. The method of any one of embodiments 1 to 20, wherein the treatment has a disease control rate of at least 98%.

Embodiment 22. The method of any one of embodiments 1 to 21, wherein the treatment has a disease control rate of at least 99%.

Embodiment 23. The method of any one of embodiments 5 to 22, wherein Compound 1, or an equivalent dose of a pharmaceutically acceptable salt thereof, is administered at a dose of about 400 mg to about 600 mg daily.

Embodiment 24. The method of any one of embodiments 5 to 22, wherein Compound 1, or an equivalent dose of a pharmaceutically acceptable salt thereof, is administered in a total daily dosage of about 400 mg to about 600 mg.

Embodiment 25. The method of any one of embodiments 5 to 24, wherein Compound 1, or an equivalent dose of a pharmaceutically acceptable salt thereof, is administered daily.

Embodiment 26. The method of any one of embodiments 5 to 25, wherein Compound 1, or an equivalent dose of a pharmaceutically acceptable salt thereof, is administered at a dose of about 300 mg two times per day (BID).

Embodiment 27. The method of any one of embodiments 5 to 25, wherein Compound 1, or an equivalent dose of a pharmaceutically acceptable salt thereof, is administered at a dose of about 200 mg two times per day (BID).

Embodiment 28. The method of any one of embodiments 6 to 27, wherein Compound 2, or an equivalent dose of a pharmaceutically acceptable salt thereof, is administered at a dose of about 400 mg to about 500 mg daily.

Embodiment 29. The method of any one of embodiments 6 to 27, wherein Compound 2, or an equivalent dose of a pharmaceutically acceptable salt thereof, is administered at a total daily dosage of about 400 mg to about 500 mg.

Embodiment 30. The method of any one of embodiments 6 to 29, wherein Compound 2, or an equivalent dose of a pharmaceutically acceptable salt thereof, is administered daily.

Embodiment 31. The method of any one of embodiments 6 to 30, wherein Compound 2, or an equivalent dose of a pharmaceutically acceptable salt thereof, is administered at a dose of about 200 mg two times per day (BID).

Embodiment 32. The method of any one of embodiments 6 to 30, wherein Compound 2, or an equivalent dose of a pharmaceutically acceptable salt thereof, is administered at a dose of about 250 mg two times per day (BID).

Embodiment 33. The method of any one of embodiments 6 to 26 and 28 to 30, wherein
Compound 1, or an equivalent dose of a pharmaceutically acceptable salt thereof, is administered at a dose of about 300 mg BID every day of at least one 7-day dosing cycle; and
Compound 2, or an equivalent dose of a pharmaceutically acceptable salt thereof, is administered at a dose of about 200 mg BID every day of at least one 7-day dosing cycle.

Embodiment 34. The method of any one of embodiments 6 to 26 and 28 to 30, wherein
Compound 1, or an equivalent dose of a pharmaceutically acceptable salt thereof, is administered daily at a dose of about 300 mg BID for at least one 7-day dosing cycle; and
Compound 2, or an equivalent dose of a pharmaceutically acceptable salt thereof, is administered daily at a dose of about 200 mg BID for at least one 7-day dosing cycle.

Embodiment 35. The method of any one of embodiments 6 to 25 and 27 to 30, wherein
Compound 1, or an equivalent dose of a pharmaceutically acceptable salt thereof, is administered at a dose of about 200 mg BID every day of at least one 7-day dosing cycle; and
Compound 2, or an equivalent dose of a pharmaceutically acceptable salt thereof, is administered at a dose of about 200 mg BID every day of at least one 7-day dosing cycle.

Embodiment 36. The method of any one of embodiments 33 to 35, wherein Compound 1, or an equivalent dose of a pharmaceutically acceptable salt thereof, and Compound 2, or an equivalent dose of a pharmaceutically acceptable salt thereof, are each administered for at least seven (7) 7-day dosing cycles.

Embodiment 37. The method of any one of embodiments 33 to 35, wherein Compound 1, or an equivalent dose of a pharmaceutically acceptable salt thereof, and Compound 2, or an equivalent dose of a pharmaceutically acceptable salt thereof, are each administered for at least eight (8) 7-day dosing cycles, at least twelve (12) 7-day dosing cycles, at least sixteen (16) 7-day dosing cycles, at least twenty (20) 7-day dosing cycles, at least twenty four (24) 7-day dosing cycles, or at least twenty eight (28) 7-day dosing cycles.

Embodiment 38. The method of any one of embodiments 6 to 26 and 28 to 30, wherein the treatment is according to a dosing schedule comprising
(i) a first treatment cycle of at least one 7-day dosing cycle, wherein Compound 1, or an equivalent dose of a pharmaceutically acceptable salt thereof, is administered alone at a dose of 300 mg BID every day of the first treatment cycle, followed by
(ii) a second treatment cycle of at least one 7-day dosing cycle, wherein Compound 1, or an equivalent dose of a pharmaceutically acceptable salt thereof, is administered at a dose of 300 mg BID, and Compound 2, or an equivalent dose of a pharmaceutically acceptable salt thereof, is administered at a dose of 200 mg BID every day of the second treatment cycle.

Embodiment 39. The method of any one of embodiments 6 to 26 and 28 to 30, wherein the treatment is according to a dosing schedule comprising
(i) a first treatment cycle of at least one 7-day dosing cycle, wherein Compound 1, or an equivalent dose of a pharmaceutically acceptable salt thereof, is administered alone at a dose of about 300 mg BID every day of the first treatment cycle, followed by
(ii) a second treatment cycle of at least three (3) 7-day dosing cycles, wherein Compound 1, or an equivalent dose of a pharmaceutically acceptable salt thereof, is administered at a dose of about 300 mg BID, and Compound 2, or an equivalent dose of a pharmaceutically acceptable salt thereof, is administered at a dose of about 200 mg BID every day of the second treatment cycle.

Embodiment 40. The method of any one of embodiments 6 to 25 and 27 to 30, wherein the treatment is according to a dosing schedule comprising
(iii) a first treatment cycle of at least one 7-day dosing cycle, wherein Compound 1, or an equivalent dose of a pharmaceutically acceptable salt thereof, is administered alone at a dose of 200 mg BID every day of the first treatment cycle, followed by
(iv) a second treatment cycle of at least one 7-day dosing cycle, wherein Compound 1, or an equivalent dose of a pharmaceutically acceptable salt thereof, is administered at a dose of 200 mg BID, and Compound 2, or an equivalent dose of a pharmaceutically acceptable salt thereof, is administered at a dose of 200 mg BID every day of the second treatment cycle.

Embodiment 41. The method of any one of embodiments 6 to 25 and 27 to 30, wherein the treatment is according to a dosing schedule comprising
(iii) a first treatment cycle of at least one 7-day dosing cycle, wherein Compound 1, or an equivalent dose of a pharmaceutically acceptable salt thereof, is administered alone at a dose of about 200 mg BID every day of the first treatment cycle, followed by
(iv) a second treatment cycle of at least three (3) 7-day dosing cycles, wherein Compound 1, or an equivalent dose of a pharmaceutically acceptable salt thereof, is administered at a dose of about 200 mg BID, and Compound 2, or an equivalent dose of a pharmaceutically acceptable salt thereof, is administered at a dose of about 200 mg BID every day of the second treatment cycle.

Embodiment 42. The method of any one of embodiments 38 to 41, wherein the second treatment cycle comprises at least seven (7) 7-day dosing cycles, at least eleven (11) 7-day dosing cycles, at least fifteen (15) 7-day dosing cycles, at least nineteen (19) 7-day dosing cycles, at least twenty-three (23) 7-day dosing cycles, or at least twenty seven (27) 7-day dosing cycles.

Embodiment 43. The method of any one of embodiments 5 to 42, wherein the treatment is uninterrupted.

Embodiment 44. The method of any one of embodiments 1 to 43, wherein the metastatic uveal melanoma is a solid tumor.

Embodiment 45. The method of embodiment 44, wherein the solid tumor harbors a GNAQ or GNA11 mutation.

Embodiment 46. The method of any one of embodiments 6 to 45, wherein Compound 1, or an equivalent dose of a pharmaceutically acceptable salt thereof, and Compound 2, or an equivalent dose of a pharmaceutically acceptable salt thereof, are administered in a single formulation.

Embodiment 47. The method of any one of embodiments 6 to 45, wherein Compound 1, or an equivalent dose of a pharmaceutically acceptable salt thereof, and Compound 2, or an equivalent dose of a pharmaceutically acceptable salt thereof, are administered in a single formulation further comprising one or more pharmaceutically acceptable carriers.

Embodiment 48. The method of any one of embodiments 6 to 45, wherein Compound 1, or an equivalent dose of a pharmaceutically acceptable salt thereof, and Compound 2, or an equivalent dose of a pharmaceutically acceptable salt thereof, are administered separately.

Embodiment 49. The method of any one of embodiments 3 to 48, wherein the screening test is selected from CT scan, MRI, PET scan, ultrasound, and X-ray, or a combination thereof.

Embodiment 50. The method of embodiment 49, wherein the screening test is CT scan.

Embodiment 51. The method of embodiment 49, wherein the screening test is MRI.

Embodiment 52. The method of embodiment 49, wherein the screening test is ultrasound.

Embodiment 53. The method of any one of embodiments 9 to 52, wherein reduction in the size of the hepatic tumor is measured using RECIST 1.1 criteria.

Embodiment 54. The method of any one of embodiments 9 to 52, wherein the size of the hepatic tumor is measured using CT scan, MRI, ultrasound, or a combination thereof.

Embodiment 55. The method of any one of embodiments 1 to 54, wherein the patient is human leukocyte antigen (HLA)-A2 negative.

Embodiment 56. The method of any one of embodiments 1 to 54, wherein the patient is human leukocyte antigen (HLA)-A2 positive.

Embodiment 57. The method of any one of embodiments 1 to 54, wherein the patient has BRCA1-associated protein-1 (BAP1) mutation.

Embodiment 58. The method of any one of embodiments 1 to 54, wherein the patient does not have BRCA1-associated protein-1 (BAP1) mutation.

Embodiment 59. The method of any one of embodiments 1 to 54, wherein the patient has normal lactate dehydrogenase (LDH) level.

Embodiment 60. The method of any one of embodiments 1 to 54, wherein the patient has higher lactate dehydrogenase (LDH) level compared to normal LDH level.

Embodiment 61. The method of any one of embodiments 1 to 60, wherein the patient is in recognized need of such treatment.

Embodiment 62. The method of any one of embodiments 1 to 61, wherein the PKC inhibitor, or a pharmaceutically acceptable salt thereof, and the cMet inhibitor, or a pharmaceutically acceptable salt thereof, are each administered orally.

### Additional Non-Limiting Exemplary Embodiments

Embodiment 1A. A PKC inhibitor for use in a method of treating metastatic uveal melanoma (MUM) in a patient having a hepatic tumor and an essential absence of an extra-hepatic tumor, the method comprising administering a therapeutically effective amount of the PKC inhibitor and administering a therapeutically effective amount of a cMet inhibitor to the patient.

Embodiment 1B. A PKC inhibitor for use in treating metastatic uveal melanoma (MUM) in a patient having a hepatic tumor and an essential absence of an extra-hepatic tumor, wherein a therapeutically effective amount of the PKC inhibitor is administered to the patient in combination with a therapeutically effective amount of a cMet inhibitor.

Embodiment 1C. Use of a PKC inhibitor in the manufacture of a medicament for the treatment of metastatic uveal melanoma (MUM) in a patient having a hepatic tumor and an essential absence of an extra-hepatic tumor, wherein a therapeutically effective amount of the PKC inhibitor is used in combination with a therapeutically effective amount of a cMet inhibitor.

Embodiment 1D. A use of a PKC inhibitor for treating metastatic uveal melanoma (MUM) in a patient having a hepatic tumor and an essential absence of an extra-hepatic tumor, wherein a therapeutically effective amount of the PKC inhibitor is used in combination with a therapeutically effective amount of a cMet inhibitor.

Embodiment 2A. A PKC inhibitor for use in a method of treating metastatic uveal melanoma (MUM) in a patient, the method comprising:
a) selecting a patient having a hepatic tumor and an essential absence of an extra-hepatic tumor; and
b) administering a therapeutically effective amount of the PKC inhibitor and administering a therapeutically effective amount of a cMet inhibitor to the patient.

Embodiment 2B. A PKC inhibitor for use in treating metastatic uveal melanoma (MUM) in a patient, wherein:
a) a patient having a hepatic tumor and an essential absence of an extra-hepatic tumor is selected; and
b) a therapeutically effective amount of the PKC inhibitor is administered to the patient in combination with a therapeutically effective amount of a cMet inhibitor.

Embodiment 2C. Use of a PKC inhibitor in the manufacture of a medicament for the treatment of metastatic uveal melanoma (MUM) in a patient, wherein:
a) a patient having a hepatic tumor and an essential absence of an extra-hepatic tumor is selected; and
b) a therapeutically effective amount of the PKC inhibitor is used in combination with a therapeutically effective amount of a cMet inhibitor.

Embodiment 2D. A use of a PKC inhibitor for treating metastatic uveal melanoma (MUM) in a patient, wherein:
a) a patient having a hepatic tumor and an essential absence of an extra-hepatic tumor is selected; and
b) a therapeutically effective amount of the PKC inhibitor is used in combination with a therapeutically effective amount of a cMet inhibitor.

Embodiment 3. The use of embodiments 1A to 1D and 2A to 2D, wherein the patient is selected by assessing a screening test.

Embodiment 4A. A PKC inhibitor for use in a method of treating metastatic uveal melanoma (MUM) in a patient, the method comprising:
a) selecting a patient having a hepatic tumor and essential absence of an extra-hepatic tumor, wherein the patient selection is determined by assessing a screening test; and
b) administering a therapeutically effective amount of the PKC inhibitor and a therapeutically effective amount of a cMet inhibitor to the patient.

Embodiment 4B. A PKC inhibitor for use in treating metastatic uveal melanoma (MUM) in a patient, wherein:
a) a patient having a hepatic tumor and essential absence of an extra-hepatic tumor is selected by assessing a screening test; and
b) a therapeutically effective amount of the PKC inhibitor is administered to the patient in combination with a therapeutically effective amount of a cMet inhibitor.

Embodiment 4C. Use of a PKC inhibitor in the manufacture of a medicament for the treatment of metastatic uveal melanoma (MUM) in a patient, wherein:
a) a patient having a hepatic tumor and essential absence of an extra-hepatic tumor is selected by assessing a screening test; and
b) a therapeutically effective amount of the PKC inhibitor is used in combination with a therapeutically effective amount of a cMet inhibitor.

Embodiment 4D. A use of a PKC inhibitor for treating metastatic uveal melanoma (MUM) in a patient, wherein:
a) a patient having a hepatic tumor and essential absence of an extra-hepatic tumor is selected by assessing a screening test; and
b) a therapeutically effective amount of the PKC inhibitor is used in combination with a therapeutically effective amount of a cMet inhibitor.

Embodiment 5. The use of any one of embodiments 1A to 4D, wherein the PKC inhibitor is Compound 1:

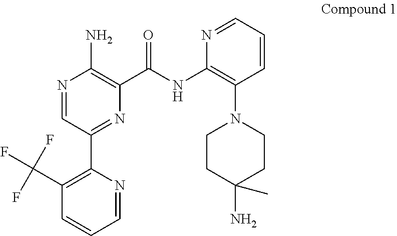 [SMILES: CC1(N)CCN(c2cccnc2NC(=O)c2nc(-c3ncccc3C(F)(F)F)cnc2N)CC1]

or a pharmaceutically acceptable salt thereof.

Embodiment 6. The use of any one of embodiments 1A to 5, wherein the cMet inhibitor is Compound 2:

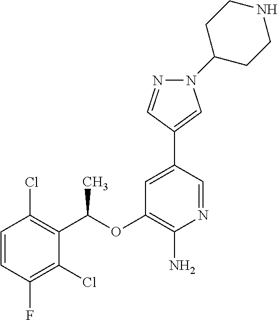 [SMILES: C[C@@H](Oc1cc(-c2cnn(C3CCNCC3)c2)cnc1N)c1c(Cl)ccc(F)c1Cl]

or a pharmaceutically acceptable salt thereof.

Embodiment 7. The use of any one of embodiments 1A to 6, wherein the patient does not have an extra-hepatic tumor.

Embodiment 8. The use of any one of embodiments 1A to 7, wherein the patient is an adult of at least 18 years of age.

Embodiment 9. The use of any one of embodiments 1A to 8, wherein the treatment reduces a size of the hepatic tumor by at least 10%.

Embodiment 10. The use of any one of embodiments 1A to 8, wherein the treatment reduces a size of the hepatic tumor by at least about 10%.

Embodiment 11. The use of any one of embodiments 1A to 10, wherein the patient has a progression free survival of greater than or equal to 3 months.

Embodiment 12. The use of any one of embodiments 1A to 11, wherein the patient has a progression free survival of greater than or equal to 4, 5, 6, 7, or 8 months.

Embodiment 13. The use of any one of embodiments 1A to 12, wherein the patient has a progression free survival of greater than or equal to 9 months.

Embodiment 14. The use of any one of embodiments 1A to 13, wherein the patient has a progression free survival of greater than or equal to 10 months.

Embodiment 15. The use of any one of embodiments 1A to 14, wherein the patient has a progression free survival of greater than or equal to 11 months.

Embodiment 16. The use of any one of embodiments 1A to 15, wherein the treatment has a disease control rate of at least 60%, 70%, or 80%.

Embodiment 17. The use of any one of embodiments 1A to 16, wherein the treatment has a disease control rate of at least 85%.

Embodiment 18. The use of any one of embodiments 1A to 17, wherein the treatment has a disease control rate of at least 90%.

Embodiment 19. The use of any one of embodiments 1A to 18, wherein the treatment has a disease control rate of at least 92%.

Embodiment 20. The use of any one of embodiments 1A to 19, wherein the treatment has a disease control rate of at least 95%.

Embodiment 21. The use of any one of embodiments 1A to 20, wherein the treatment has a disease control rate of at least 98%.

Embodiment 22. The use of any one of embodiments 1A to 21, wherein the treatment has a disease control rate of at least 99%.

Embodiment 23. The use of any one of embodiments 5 to 22, wherein Compound 1, or an equivalent dose of a pharmaceutically acceptable salt thereof, is administered at a dose of about 400 mg to about 600 mg daily.

Embodiment 24. The use of any one of embodiments 5 to 22, wherein Compound 1, or an equivalent dose of a pharmaceutically acceptable salt thereof, is administered in a total daily dosage of about 400 mg to about 600 mg.

Embodiment 25. The use of any one of embodiments 5 to 24, wherein Compound 1, or an equivalent dose of a pharmaceutically acceptable salt thereof, is administered daily.

Embodiment 26. The use of any one of embodiments 5 to 25, wherein Compound 1, or an equivalent dose of a pharmaceutically acceptable salt thereof, is administered at a dose of about 300 mg two times per day (BID).

Embodiment 27. The use of any one of embodiments 5 to 25, wherein Compound 1, or an equivalent dose of a pharmaceutically acceptable salt thereof, is administered at a dose of about 200 mg two times per day (BID).

Embodiment 28. The use of any one of embodiments 6 to 27, wherein Compound 2, or an equivalent dose of a pharmaceutically acceptable salt thereof, is administered at a dose of about 400 mg to about 500 mg daily.

Embodiment 29. The use of any one of embodiments 6 to 27, wherein Compound 2, or an equivalent dose of a pharmaceutically acceptable salt thereof, is administered at a total daily dosage of about 400 mg to about 500 mg.

Embodiment 30. The use of any one of embodiments 6 to 29, wherein Compound 2, or an equivalent dose of a pharmaceutically acceptable salt thereof, is administered daily.

Embodiment 31. The use of any one of embodiments 6 to 30, wherein Compound 2, or an equivalent dose of a pharmaceutically acceptable salt thereof, is administered at a dose of about 200 mg two times per day (BID).

Embodiment 32. The use of any one of embodiments 6 to 30, wherein Compound 2, or an equivalent dose of a pharmaceutically acceptable salt thereof, is administered at a dose of about 250 mg two times per day (BID).

Embodiment 33. The use of any one of embodiments 6 to 26 and 28 to 30, wherein
Compound 1, or an equivalent dose of a pharmaceutically acceptable salt thereof, is administered at a dose of about 300 mg BID every day of at least one 7-day dosing cycle; and
Compound 2, or an equivalent dose of a pharmaceutically acceptable salt thereof, is administered at a dose of about 200 mg BID every day of at least one 7-day dosing cycle.

Embodiment 34. The use of any one of embodiments 6 to 26 and 28 to 30, wherein
Compound 1, or an equivalent dose of a pharmaceutically acceptable salt thereof, is administered daily at a dose of about 300 mg BID for at least one 7-day dosing cycle; and
Compound 2, or an equivalent dose of a pharmaceutically acceptable salt thereof, is administered daily at a dose of about 200 mg BID for at least one 7-day dosing cycle.

Embodiment 35. The use of any one of embodiments 6 to 25 and 27 to 30, wherein
Compound 1, or an equivalent dose of a pharmaceutically acceptable salt thereof, is administered at a dose of about 200 mg BID every day of at least one 7-day dosing cycle; and
Compound 2, or an equivalent dose of a pharmaceutically acceptable salt thereof, is administered at a dose of about 200 mg BID every day of at least one 7-day dosing cycle.

Embodiment 36. The use of any one of embodiments 33 to 35, wherein Compound 1, or an equivalent dose of a pharmaceutically acceptable salt thereof, and Compound 2, or an equivalent dose of a pharmaceutically acceptable salt thereof, are each administered for at least seven (7) 7-day dosing cycles.

Embodiment 37. The use of any one of embodiments 33 to 35, wherein Compound 1, or an equivalent dose of a pharmaceutically acceptable salt thereof, and Compound 2, or an equivalent dose of a pharmaceutically acceptable salt thereof, are each administered for at least eight (8) 7-day dosing cycles, at least twelve (12) 7-day dosing cycles, at least sixteen (16) 7-day dosing cycles, at least twenty (20) 7-day dosing cycles, at least twenty four (24) 7-day dosing cycles, or at least twenty eight (28) 7-day dosing cycles.

Embodiment 38. The use of any one of embodiments 6 to 26 and 28 to 30, wherein the treatment is according to a dosing schedule comprising
(v) a first treatment cycle of at least one 7-day dosing cycle, wherein Compound 1, or an equivalent dose of a pharmaceutically acceptable salt thereof, is administered alone at a dose of 300 mg BID every day of the first treatment cycle, followed by
(vi) a second treatment cycle of at least one 7-day dosing cycle, wherein Compound 1, or an equivalent dose of a pharmaceutically acceptable salt thereof, is administered at a dose of 300 mg BID, and Compound 2, or an equivalent dose of a pharmaceutically acceptable salt thereof, is administered at a dose of 200 mg BID every day of the second treatment cycle.

Embodiment 39. The use of any one of embodiments 6 to 26 and 28 to 30, wherein the treatment is according to a dosing schedule comprising
(v) a first treatment cycle of at least one 7-day dosing cycle, wherein Compound 1, or an equivalent dose of a pharmaceutically acceptable salt thereof, is administered alone at a dose of about 300 mg BID every day of the first treatment cycle, followed by
(vi) a second treatment cycle of at least three (3) 7-day dosing cycles, wherein Compound 1, or an equivalent dose of a pharmaceutically acceptable salt thereof, is administered at a dose of about 300 mg BID, and Compound 2, or an equivalent dose of a pharmaceutically acceptable salt thereof, is administered at a dose of about 200 mg BID every day of the second treatment cycle.

Embodiment 40. The use of any one of embodiments 6 to 25 and 27 to 30, wherein the treatment is according to a dosing schedule comprising
(vii) a first treatment cycle of at least one 7-day dosing cycle, wherein Compound 1, or an equivalent dose of a pharmaceutically acceptable salt thereof, is administered alone at a dose of 200 mg BID every day of the first treatment cycle, followed by
(viii) a second treatment cycle of at least one 7-day dosing cycle, wherein Compound 1, or an equivalent dose of a pharmaceutically acceptable salt thereof, is administered at a dose of 200 mg BID, and Compound 2, or an equivalent dose of a pharmaceutically acceptable salt thereof, is administered at a dose of 200 mg BID every day of the second treatment cycle.

Embodiment 41. The use of any one of embodiments 6 to 25 and 27 to 30, wherein the treatment is according to a dosing schedule comprising
(vii) a first treatment cycle of at least one 7-day dosing cycle, wherein Compound 1, or an equivalent dose of a pharmaceutically acceptable salt thereof, is administered alone at a dose of about 200 mg BID every day of the first treatment cycle, followed by
(viii) a second treatment cycle of at least three (3) 7-day dosing cycles, wherein Compound 1, or an equivalent dose of a pharmaceutically acceptable salt thereof, is administered at a dose of about 200 mg BID, and Compound 2, or an equivalent dose of a pharmaceutically acceptable salt thereof, is administered at a dose of about 200 mg BID every day of the second treatment cycle.

Embodiment 42. The use of any one of embodiments 38 to 41, wherein the second treatment cycle comprises at least seven (7) 7-day dosing cycles, at least eleven (11) 7-day dosing cycles, at least fifteen (15) 7-day dosing cycles, at least nineteen (19) 7-day dosing cycles, at least twenty-three (23) 7-day dosing cycles, or at least twenty seven (27) 7-day dosing cycles.

Embodiment 43. The use of any one of embodiments 5 to 42, wherein the treatment is uninterrupted.

Embodiment 44. The use of any one of embodiments 1A to 43, wherein the metastatic uveal melanoma is a solid tumor.

Embodiment 45. The use of embodiment 44, wherein the solid tumor harbors a GNAQ or GNA11 mutation.

Embodiment 46. The use of any one of embodiments 6 to 45, wherein Compound 1, or an equivalent dose of a pharmaceutically acceptable salt thereof, and Compound 2, or an equivalent dose of a pharmaceutically acceptable salt thereof, are administered in a single formulation.

Embodiment 47. The use of any one of embodiments 6 to 45, wherein Compound 1, or an equivalent dose of a pharmaceutically acceptable salt thereof, and Compound 2, or an equivalent dose of a pharmaceutically acceptable salt thereof, are administered in a single formulation further comprising one or more pharmaceutically acceptable carriers.

Embodiment 48. The use of any one of embodiments 6 to 45, wherein Compound 1, or an equivalent dose of a pharmaceutically acceptable salt thereof, and Compound 2, or an equivalent dose of a pharmaceutically acceptable salt thereof, are administered separately.

Embodiment 49. The use of any one of embodiments 3 to 48, wherein the screening test is selected from CT scan, MRI, PET scan, ultrasound, and X-ray, or a combination thereof.

Embodiment 50. The use of embodiment 49, wherein the screening test is CT scan.

Embodiment 51. The use of embodiment 49, wherein the screening test is MRI.

Embodiment 52. The use of embodiment 49, wherein the screening test is ultrasound.

Embodiment 53. The use of any one of embodiments 9 to 52, wherein reduction in the size of the hepatic tumor is measured using RECIST 1.1 criteria.

Embodiment 54. The use of any one of embodiments 9 to 52, wherein the size of the hepatic tumor is measured using CT scan, MRI, ultrasound, or a combination thereof.

Embodiment 55. The use of any one of embodiments 1A to 54, wherein the patient is human leukocyte antigen (HLA)-A2 negative.

Embodiment 56. The use of any one of embodiments 1A to 54, wherein the patient is human leukocyte antigen (HLA)-A2 positive.

Embodiment 57. The use of any one of embodiments 1A to 54, wherein the patient has BRCA1-associated protein-1 (BAP1) mutation.

Embodiment 58. The use of any one of embodiments 1A to 54, wherein the patient does not have BRCA1-associated protein-1 (BAP1) mutation.

Embodiment 59. The use of any one of embodiments 1A to 54, wherein the patient has normal lactate dehydrogenase (LDH) level.

Embodiment 60. The use of any one of embodiments 1A to 54, wherein the patient has higher lactate dehydrogenase (LDH) level compared to normal LDH level.

Embodiment 61. The use of any one of embodiments 1A to 60, wherein the patient is in recognized need of such treatment.

Embodiment 62. The use of any one of embodiments 1 to 61, wherein the PKC inhibitor, or a pharmaceutically acceptable salt thereof, and the cMet inhibitor, or a pharmaceutically acceptable salt thereof, are each administered orally.

For the embodiments above, when reference is made to previous embodiments, the reference will include those embodiments having lettered notations or combinations. For example, reference to embodiments 1A to 7 will include embodiments 1A, 1B, 1C, 1D, 2A, 2B, 2C, 3D, 3, 4A, 4B, 4C, 4D, 5, 6, and 7.

### EXAMPLES

The compounds and methods disclosed herein are further illustrated by the following examples, which should not be construed as further limiting. The practice of the present disclosure will employ, unless otherwise indicated, conventional techniques of organic synthesis, cell biology, cell culture, and molecular biology, which are within the skill of the art.

Processes for preparing the compounds disclosed herein can be found, at least, in WO 2016/020864 and WO 2006/021884, the contents of which are incorporated in their entirety.

### Example 1: Clinical Data

Provided below is clinical trial (NCT #03947385) data for patients receiving Compound 1 and Compound 2 to treat hepatic-only metastatic uveal melanoma (MUM).

TABLE 1

| Response by RECIST 1.1 in | Evaluable |
|---|---|
| Hepatic-Only MUM | Patients (N = 20) |
| Confirmed ORR (7/20) | 35% |
| Tumor Shrinkage (20/20) | 100% |
| >30% Tumor Shrinkage (9/20) | 45% |

Best Overall Response

| | |
|---|---|
| cPR (7/20) | 35% |
| uPR (1/20) | 5% |
| SD (12/20) | 60% |
| DCR (20/20) | 100% |

In this trial, for heptic-only MUM patients (N = 20/20), the mPFS is ~11 months.
“N” refers to the number of patients.

TABLE 2

| | Tumor size from |
|---|---|
| | baseline (determined |
| | from post baseline scan) |
| | (Best of % Change |
| Patient ID | RECIST 1.1) |
| 1 | −3% |
| 2 | −9% |
| 3 | −12% |
| 4 | −13% |
| 5 | −17% |
| 6 | −17% |
| 7 | −17% |
| 8 | −22% |
| 9 | −24% |
| 10 | −25% |
| 11 | −26% |
| 12 | −33% |
| 13 | −35% |
| 14 | −36% |
| 15 | −37% |
| 16 | −40% |
| 17 | −42% |
| 18 | −48% |
| 19 | −65% |
| 20 | −69% |

Note (Table 2):
All patients were dosed with Compound 1 at 300 mg BID and Compound 2 at 200 mg BID for at least 56 days (8 weeks).
“N” refers to the number of patients.

### Example 1A: Updates to Example 1

Provided below is clinical trial (NCT #03947385) data for patients receiving Compound 1 and Compound 2 to treat hepatic-only metastatic uveal melanoma (MUM) as of Apr. 11, 2024.

TABLE 3

| Response by RECIST 1.1 in | Evaluable* |
|---|---|
| Hepatic-Only MUM | Patients (N = 29) |
| Confirmed ORR (10/29) | 34.5% |
| Tumor Shrinkage (26/29) | 89.7% |
| >30% Tumor Shrinkage (14/29) | 48.3% |

Best Overall Response

| | |
|---|---|
| cPR (10/29) | 34.5% |
| uPR (0/29) | 0.0% |
| SD (18/29) | 62.1% |
| DCR (28/29) | 96.6% |

*Evaluable defined as having at least 1 dose of study drug, measurable tumor at baseline and 1 post-baseline scan.

In this trial, for hepatic-only MUM patients (N=29), the mPFS is ~7.6 months [95% CI: 5, 13.6] with a median duration of response (mDOR) (n=10) of 9.0 months [95% CI: 1.9, 15.9].

TABLE 4

| | Tumor size from |
|---|---|
| | baseline (determined |
| | from post baseline scan) |
| | (Best of % Change |
| Patient ID | RECIST 1.1) |
| 1 | −3% |
| 2 | −9% |
| 4 | −57% |
| 6 | −17% |
| 7 | −28% |
| 8 | −22% |
| 9 | −53% |
| 10 | −25% |
| 11 | −26% |
| 12 | −33% |
| 13 | −35% |
| 14 | −36% |
| 15 | −37% |
| 16 | −40% |
| 17 | −50% |
| 18 | −48% |
| 19 | −65% |
| 20 | −69% |
| 21 | −69% |
| 22 | −29% |
| 23 | −25% |
| 24 | −33% |
| 25 | −5% |
| 26 | +5% |
| 27 | 0% |
| 28 | +23% |
| 29 | −27% |
| 30 | −26% |
| 31 | +28% |
| 32 | −12% |

Note (Table 4):
All evaluable hepatic-only MUM patients were dosed with Compound 1 at 300 mg BID and Compound 2 at 200 mg BID for at least 84 days (Range 84-948 days)

As of Apr. 11, 2024, eight patients were dosed with Compound 1 at 200 mg BID and Compound 2 at 200 mg BID. See Table 5 and Table 6 below.

TABLE 5

| Response by RECIST 1.1 in | Evaluable* |
|---|---|
| Hepatic-Only MUM | Patients (N = 8) |
| Confirmed ORR (1/8) | 12.5% |
| Tumor Shrinkage (7/8) | 87.5% |
| >30% Tumor Shrinkage (1/8) | 12.5% |

Best Overall Response

| | |
|---|---|
| cPR (1/8) | 12.5% |
| uPR (0/8) | 0% |
| SD (7/8) | 87.5% |
| DCR (8/8) | 100% |

*Evaluable defined as having at least 1 dose of study drug, measurable tumor at baseline and 1 post-baseline scan.
“N” refers to the number of patients.

In this trial, for hepatic-only MUM patients (N=8) (Compound 1 at 200 mg BID and Compound 2 at 200 mg BID), the mPFS is ~5.1 months [95% CI: 3.7, −]. The “−” indicates that upper bound of the CI has not yet been determined. “CI” refers to confidence interval.

TABLE 6

| | Tumor size from |
|---|---|
| | baseline (determined |
| | from post baseline scan) |
| | (Best of % Change |
| Patient ID | RECIST 1.1) |
| 33 | −29% |
| 34 | −19% |
| 35 | −17% |
| 36 | −3% |
| 37 | −46% |
| 38 | +4% |
| 39 | −25% |
| 40 | −22% |

Note (Table 6):
All patients were dosed with Compound 1 at 200 mg BID and Compound 2 at 200 mg BID for at least 112 days [Range 112-458 days].

Safety Summary: Most common adverse events (AEs) (n=39; all grade, in ≥20% of pts) suspected related to Compound 1 and Compound 2 combination treatment in patients across both schedules (n=39) were diarrhea (92.3%), nausea (82.1%), dermatitis acneiform (59%), oedema peripheral (56.4%), vomiting (43.6%), hypoalbuminemia (35.9%), dizziness (33.3%), fatigue (30.8%), anemia (20.5%), constipation (20.5%), hypotension (20.5%). The majority of GI and constitutional AEs were grade (gr) ½. Gr ¾ AEs suspected related to Compound 1 and Compound 2 combination treatment were reported in 8 pts (20.5%), the most common being Grade 3 syncope (10.3%) and Grade 3 anemia (5.1%) and Electrocardiogram QT prolongation (5.1%) and 1 case of Grade 4 Neutropenia (2.6%). All other gr 3 AEs occurred in 1 patient (2.6%) each. There were no Gr 5 AEs.

A liver clamp invasive procedure using melphalan shows the DCR of 76.3% (Zager J S, Orloff M M, Ferrucci P F, et al.: FOCUS phase 3 trial results: Percutaneous hepatic perfusion (PHP) with melphalan for patients with ocular melanoma liver metastases (PHP-OCM-301/301A). J Clin Oncol 40:9510, 2022), which is low compared to the Compound 1 and Compound 2 combination oral treatment.

The clinical trial (NCT #03947385) referred herein was conducted under the following patient inclusion and exclusion criteria:

### Inclusion Criteria:

Patient must be ≥18 years of age
Diagnosis of one of the following:
   MUM: Uveal melanoma with histological or cytological confirmed metastatic disease.
Measurable disease
Eastern Cooperative Oncology Group≤1 and expected life expectancy of >3 months
Adequate organ function at screening
Adequate contraceptive measures for non-sterilized male and female patients of childbearing potential

### Crizotinib Combination Additional Inclusion Criteria:

Prior chemotherapy other therapies as applicable or major surgeries must have been completed at least 4 weeks prior to initiation of crizotinib
Patients with preexisting peripheral neuropathy can be included if it is Grade 1 or lower, prior to initiation of crizotinib

### Exclusion Criteria:

Known symptomatic brain metastases
Previous treatment with a PKC inhibitor
Known MSI-H/dMMR tumors who have not previously received immune checkpoint inhibitors
Adverse events from prior anti-cancer therapy that have not resolved
Known acquired immunodeficiency syndrome (AIDS)-related illness, hepatitis B virus, or hepatitis C virus
Active infection requiring ongoing therapy
Recent surgery or radiotherapy
Prior gastrectomy or upper bowel removal or any other gastrointestinal disorder or defect
Females who are pregnant or breastfeeding
Impaired cardiac function
Treatment with prohibited medications that cannot be discontinued prior to study entry
For patients receiving IDE196 powder-in-capsule (PIC) formulation or crizotinib, allergy to mammalian meat products and gelatin

### Crizotinib Combination Additional Exclusion Criteria:

Prior therapy directly targeting ALK, MET, or ROS1
Spinal cord compression
History of pneumonitis or interstitial lung disease
History of syncope

Particular embodiments of this invention are described herein, including the best mode known to the inventors for carrying out the invention. Upon reading the foregoing, description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

1. A method of treating metastatic uveal melanoma (MUM) in a patient having a hepatic tumor and an essential absence of an extra-hepatic tumor, the method comprising administering a therapeutically effective amount of a PKC inhibitor and administering a therapeutically effective amount of a cMet inhibitor to the patient.

2. A method of treating metastatic uveal melanoma (MUM) in a patient, the method comprising:
a) selecting a patient having a hepatic tumor and an essential absence of an extra-hepatic tumor; and
b) administering a therapeutically effective amount of a PKC inhibitor and administering a therapeutically effective amount of a cMet inhibitor to the patient.

3. The method of claim 1 or 2, wherein the patient is selected by assessing a screening test.

4. A method of treating metastatic uveal melanoma (MUM) in a patient, the method comprising:
a) selecting a patient having a hepatic tumor and essential absence of an extra-hepatic tumor, wherein the patient selection is determined by assessing a screening test; and
b) administering a therapeutically effective amount of a PKC inhibitor and a therapeutically effective amount of a cMet inhibitor to the patient.

5. The method of any one of claims 1 to 4, wherein the PKC inhibitor is Compound 1: 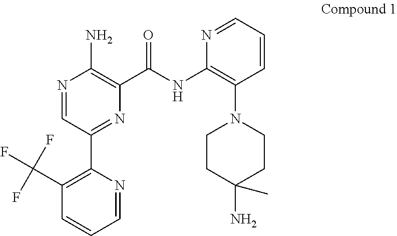 [SMILES: CC1(N)CCN(c2cccnc2NC(=O)c2nc(-c3ncccc3C(F)(F)F)cnc2N)CC1]
or a pharmaceutically acceptable salt thereof.

6. The method of any one of claims 1 to 5, wherein the cMet inhibitor is Compound 2: 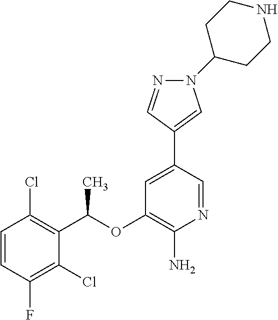 [SMILES: C[C@@H](Oc1cc(-c2cnn(C3CCNCC3)c2)cnc1N)c1c(Cl)ccc(F)c1Cl]
or a pharmaceutically acceptable salt thereof.

7. The method of any one of claims 1 to 6, wherein the patient does not have an extra-hepatic tumor.

8. The method of any one of claims 1 to 7, wherein the treatment reduces a size of the hepatic tumor by at least 10%.

9. The method of any one of claims 1 to 8, wherein the patient has a progression free survival of greater than or equal to 3, 4, 5, 6, 7, 8, or 9, months.

10. The method of any one of claims 1 to 9, wherein the patient has a progression free survival of greater than or equal to 10 or 11 months.

11. The method of any one of claims 1 to 10, wherein the treatment has a disease control rate of at least 60%, 70%, 80%, 85%, 90%, 92%, or 95%.

12. The method of any one of claims 1 to 11, wherein the treatment has a disease control rate of at least 98%.

13. The method of any one of claims 1 to 12, wherein the treatment has a disease control rate of at least 99%.

14. The method of any one of claims 5 to 13, wherein Compound 1, or an equivalent dose of a pharmaceutically acceptable salt thereof, is administered in a total daily dosage of about 400 mg to about 600 mg.

15. The method of any one of claims 5 to 14, wherein Compound 1, or an equivalent dose of a pharmaceutically acceptable salt thereof, is administered at a dose of about 300 mg two times per day (BID).

16. The method of any one of claims 5 to 14, wherein Compound 1, or an equivalent dose of a pharmaceutically acceptable salt thereof, is administered at a dose of about 200 mg two times per day (BID).

17. The method of any one of claims 5 to 16, wherein Compound 1, or an equivalent dose of a pharmaceutically acceptable salt thereof, is administered daily.

18. The method of any one of claims 6 to 17, wherein Compound 2, or an equivalent dose of a pharmaceutically acceptable salt thereof, is administered in a total daily dosage of about 400 mg to about 500 mg.

19. The method of any one of claims 6 to 18, wherein Compound 2, or an equivalent dose of a pharmaceutically acceptable salt thereof, is administered at a dose of about 200 mg two times per day (BID).

20. The method of any one of claims 6 to 15 and 17 to 19, wherein
Compound 1, or an equivalent dose of a pharmaceutically acceptable salt thereof, is administered at a dose of about 300 mg BID every day of at least one 7-day dosing cycle; and
Compound 2, or an equivalent dose of a pharmaceutically acceptable salt thereof, is administered at a dose of about 200 mg BID every day of at least one 7-day dosing cycle.

21. The method of any one of claims 6 to 14 and 16 to 19, wherein
Compound 1, or an equivalent dose of a pharmaceutically acceptable salt thereof, is administered at a dose of about 200 mg BID every day of at least one 7-day dosing cycle; and
Compound 2, or an equivalent dose of a pharmaceutically acceptable salt thereof, is administered at a dose of about 200 mg BID every day of at least one 7-day dosing cycle.

22. The method of claim 21, wherein Compound 1, or an equivalent dose of a pharmaceutically acceptable salt thereof, and Compound 2, or an equivalent dose of a pharmaceutically acceptable salt thereof, are each administered for at least eight (8) 7-day dosing cycles.

23. The method of any one of claims 6 to 15 and 17 to 20, wherein the treatment is according to a dosing schedule comprising
(i) a first treatment cycle of at least one 7-day dosing cycle, wherein Compound 1, or an equivalent dose of a pharmaceutically acceptable salt thereof, is administered alone at a dose of 300 mg BID every day of the first treatment cycle, followed by
(ii) a second treatment cycle of at least one 7-day dosing cycle, wherein Compound 1, or an equivalent dose of a pharmaceutically acceptable salt thereof, is administered at a dose of 300 mg BID, and Compound 2, or an equivalent dose of a pharmaceutically acceptable salt thereof, is administered at a dose of 200 mg BID every day of the second treatment cycle.

24. The method of any one of claims 6 to 15 and 17 to 20, wherein the treatment is according to a dosing schedule comprising
(i) a first treatment cycle of at least one 7-day dosing cycle, wherein Compound 1, or an equivalent dose of a pharmaceutically acceptable salt thereof, is administered alone at a dose of about 300 mg BID every day of the first treatment cycle, followed by
(ii) a second treatment cycle of at least three (3) 7-day dosing cycles, wherein Compound 1, or an equivalent dose of a pharmaceutically acceptable salt thereof, is administered at a dose of about 300 mg BID, and Compound 2, or an equivalent dose of a pharmaceutically acceptable salt thereof, is administered at a dose of about 200 mg BID every day of the second treatment cycle.

25. The method of any one of claims 6 to 14, 16 to 19, and 21, wherein the treatment is according to a dosing schedule comprising
(i) a first treatment cycle of at least one 7-day dosing cycle, wherein Compound 1, or an equivalent dose of a pharmaceutically acceptable salt thereof, is administered alone at a dose of 200 mg BID every day of the first treatment cycle, followed by
(ii) a second treatment cycle of at least one 7-day dosing cycle, wherein Compound 1, or an equivalent dose of a pharmaceutically acceptable salt thereof, is administered at a dose of 200 mg BID, and Compound 2, or an equivalent dose of a pharmaceutically acceptable salt thereof, is administered at a dose of 200 mg BID every day of the second treatment cycle.

26. The method of any one of claims 6 to 14, 16 to 19, and 21, wherein the treatment is according to a dosing schedule comprising
(i) a first treatment cycle of at least one 7-day dosing cycle, wherein Compound 1, or an equivalent dose of a pharmaceutically acceptable salt thereof, is administered alone at a dose of about 200 mg BID every day of the first treatment cycle, followed by
(ii) a second treatment cycle of at least three (3) 7-day dosing cycles, wherein Compound 1, or an equivalent dose of a pharmaceutically acceptable salt thereof, is administered at a dose of about 200 mg BID, and Compound 2, or an equivalent dose of a pharmaceutically acceptable salt thereof, is administered at a dose of about 200 mg BID every day of the second treatment cycle.

27. The method of any one of claims 23 to 26, wherein the second treatment cycle comprises at least seven (7) 7-day dosing cycles, at least eleven (11) 7-day dosing cycles, at least fifteen (15) 7-day dosing cycles, at least nineteen (19) 7-day dosing cycles, at least twenty-three (23) 7-day dosing cycles, or at least twenty-seven (27) 7-day dosing cycles.28.
The method of any one of claims 1 to 27, wherein the metastatic uveal melanoma is a solid tumor.

29. The method of claim 28, wherein the solid tumor harbors a GNAQ or GNA11 mutation.

30. The method of any one of claims 6 to 29, wherein Compound 1, or an equivalent dose of a pharmaceutically acceptable salt thereof, and Compound 2, or an equivalent dose of a pharmaceutically acceptable salt thereof, are administered separately.

31. The method of any one of claims 3 to 30, wherein the screening test is selected from CT scan, MRI, PET scan, ultrasound, and X-ray, or a combination thereof.

32. The method of any one of embodiments 8 to 31, wherein reduction in the size of the hepatic tumor is measured using RECIST 1.1 criteria.

33. The method of any one of claims 1 to 32, wherein the patient is in recognized need of such treatment.

34. The method of any one of claims 1 to 33, wherein the PKC inhibitor, or a pharmaceutically acceptable salt thereof, and the cMet inhibitor, or a pharmaceutically acceptable salt thereof, are each administered orally.
